(19) United States
(12) Patent Application Publication
(10) Pub. No.: US 2012/0035125 A1
(43) Pub. Date: Feb. 9, 2012

(54) FULVIC ACID IN COMBINATION WITH FLUCONAZOLE OR AMPHOTERICIN B FOR THE TREATMENT OF FUNGAL INFECTIONS

(75) Inventors: Peter Warn, Worsley Manchester (GB); Stephen Williams Leivers, Wymondham Norfolk (GB)

(73) Assignee: Natracine UK Limited, Mottram St Andrew Cheshire (GB)

(21) Appl. No.: 13/144,398

(22) PCT Filed: Jan. 18, 2010
(86) PCT No.: PCT/IB2010/050213

§ 371 Date: Sep. 22, 2011

(30) Foreign Application Priority Data
Jan. 19, 2009 (GB) 0900786.5

(51) Int. Cl. A61K 31/7048; A61P 31/10; A61K 31/4196

(52) U.S. Cl. 514/31; 514/383

(57) ABSTRACT

A combination of fulvic acid or a salt, ester, or derivative thereof and an antifungal compound selected from fluconazole and amphotericin B for use in the treatment or prophylaxis of a disease or condition of the human or animal body is described. The fulvic acid can be CHD-FA.

### BACKGROUND OF THE INVENTION

THIS invention relates to fulvic acid in combination with one or more antifungal compounds for use in the therapeutic treatment or prophylaxis of various conditions in the human or animal body.

Fulvic acid is one of the substances formed during the decay of organic matter in the environment. It is soluble in water under all pH conditions and is generally of a lower molecular size and weight and lower in colour intensity than the humic acids also produced during the decay process.

Although fulvic acid occurs naturally in low levels in soil and water, it is difficult to isolate. A known process for yielding fulvic acid for use in medicinal applications is by a controlled wet oxidation of bituminous coal as described in U.S. Pat. No. 4,912,256. The use of such fulvic acid for treatment of inflammation, acne, eczema, bacterial, fungal and viral infections has been previously disclosed in International Patent Publication WO00/19999. In addition, U.S. Pat. Nos. 4,999,202 and 5,204,368 disclose compositions containing fulvic acid, salt or a derivative thereof, which have bacteriostatic or bacteriocidal properties and are useful as disinfectants.

Coal derived fulvic acids contain high concentrations of heavy metals such as aluminium, mercury, cadmium, chromium and lead that must be avoided in pharmaceutical preparations. A fulvic acid composition derived from a carbohydrate source (CHD-FA) by wet oxidation has been previously disclosed in International Patent Publication WO2007/125492. This CHD-FA is particularly useful for pharmaceutical application in that is has a low content of heavy metals.

### SUMMARY OF THE INVENTION

According to a first embodiment of the invention, there is provided a combination of fulvic acid or a salt, ester, or derivative thereof and an antifungal compound selected from fluconazole and amphotericin B.

The fulvic acid, salt, ester or derivative thereof can have any pH, from acid to basic. For example, the pH of the fulvic acid can be raised by converting the acid into a salt, such as the sodium or potassium salt. This may be achieved by adding a suitable hydroxide to the fulvic acid. Typically, the fulvic acid is in the form of an acid or a salt.

Preferably, the fulvic acid is carbohydrate derived (CHD-FA) produced by the method described in WO 2007/125492. In particular, the CHD-FA may be derived from a saccharide. Typically, the CHD-FA has a molecular weight not exceeding 20,000 Daltons and a low content of the elements aluminium, mercury, cadmium, and chromium. The CHD-FA is manufactured by subjecting a carbohydrate to wet oxidation and then treating the reaction product obtained to remove substantially all acidic components with a molecular weight exceeding 20,000 Daltons.

Fluconazole is a known antifungal agent and is described as, for example, Item 4122 of the Merck Index, 14^{th} Ed. The fluconazole can be administered in the form of an ester and it is to be understood that the term “fluconazole” as used herein and in the claims includes esters and other suitable pharmaceutical forms of fluconazole.

Amphotericin B is also a known antifungal agent and is described as, for example, Item 585 of the Merck Index, 14^{th} Ed.

The combination of fulvic acid and fluconazole has surprisingly been found to be effective when administered against fluconazole-resistant fungi. In particular, the combination of fulvic acid and fluconazole is effective when administered against fluconazole-resistant Candida spp.

Amphotericin B has surprisingly been found to be effective at a lower, non-toxic dose when administered against fungal species in combination with fulvic acid.

In one form of the invention the combination comprises from about 10 ml/kg of a solution of about 0.25% to about 1% fulvic acid or a salt, ester or derivative thereof and about 10 mg/kg of fluconazole.

The amphotericin B may be present in the combination at an effective dose that is non-toxic to the subject. More particularly, the combination comprises about 0.25% fulvic acid and from about 0.06 mg/l to about 0.5 mg/l of amphotericin B.

According to a further embodiment of the invention there is provided a pharmaceutical composition comprising the combination as described above as the active ingredients.

The pharmaceutical composition may be in a form suitable for oral administration, or topical administration or any other suitable form of administration. For example, the pharmaceutical composition may be formulated into a liquid, tablet, capsule or the like or into a cream, or ointment.

According to a further aspect of the invention there is provided a combination or pharmaceutical composition as described above for use in a method of treatment or prophylaxis of a disease or condition of the human or animal body. The method may comprise oral, topical or any other suitable form of administration.

The human or animal to be treated may be immunosuppressed or immunocompromised.

The diseases or condition may be caused by a drug-resistant fungus.

The disease or condition may be caused by yeasts. Preferably, the disease or condition is caused by Candida spp.

The disease or condition may be caused by Aspergillus spp or Zygomycetes.

According to a further aspect of the invention is provided the use of the combination described above in the manufacture of a pharmaceutical composition for treatment or prophylaxis of a disease or condition of the human or animal body.

The pharmaceutical composition may be in a form suitable for oral administration, or topical administration or any other suitable form of administration. For example, the pharmaceutical composition may be formulated into a liquid, tablet, capsule or the like or into a cream, or ointment.

### BRIEF DESCRIPTION OF THE DRAWINGS

FIG. 1 shows kidney tissue burden following Candida albicans infection in mice treated with different concentrations of CHD-FA alone or in combination with fluconazole.

### DETAILED DESCRIPTION OF THE INVENTION

Drug resistance is a major problem for the treatment of diseases and conditions caused by fungal agents, such as has occurred with the use of fluconazole for the treatment of Candida infection and the use of amphotericin B in the treatment of Aspergillus. In particular, use of amphotericin B in the treatment of Aspergillus infections is no longer effective, as the dose required to inhibit Aspergillus is toxic to the subject (3 mg/l).

In addition, the opportunistic fungal infections that occur in immunocompromised, or immunosuppressed patients by are difficult to control with antifungal agents. A treatment strategy is therefore needed in these patients, particularly cancer patients who are receiving anti-cancer drugs and other patients on any drugs that cause immunosuppression.

Three studies were conducted to evaluate the antifungal characteristics of fulvic acid against specific organisms. The fulvic acid used in these studies was that described in, and produced by the method described in WO 2007/125492 and is hereinafter referred to as CHD-FA. In brief, the fulvic acid was derived from a carbohydrate, in particular a saccharide. The CHD-FA has a molecular weight not exceeding 20,000 Daltons and a low content, i.e. less than 30 ppm, of elements such as aluminium, mercury, cadmium, chromium, lead, silver, arsenic and beryllium. The CHD-FA was manufactured by subjecting the carbohydrate to wet oxidation and then treating the reaction product obtained to remove substantially all acidic components with a molecular weight exceeding 20,000 Daltons.

In the first study, kidney burdens of Candida albicans was determined as an indication of the efficacy of increasing concentrations of CHD-FA alone or in combination with the antifungal compound, fluconazole. Results showed that fulvic acid significantly enhances the activity of fluconazole against Candida albicans.

In the second study, the efficacy of fulvic acid alone or in combination with the antifungal compound amphotericin B against Aspergillus or zygomycetes was determined by quantitative colony counts in tissue culture plates.

In the third study, the efficacy of fluconazole in combination with fulvic acid against drug resistant strains of Candida spp was determined by quantitative colony counts in tissue culture plates.

All solutions are set out as percentages of weight per volume.

The following examples are for the purpose of illustration only and are not to be construed as limiting on the invention in any way.

### EXAMPLE 1

### In Vivo Efficacy of CHD-FA Against Candida

1.1. Physical Properties of New Antibiotic Resistance Modulating Agent Also Known as Carbohydrate Derived Fulvic Acid (CHD-FA)

CHD-FA was reconstituted as a 4% solution. The solution was stored at room temperature in the dark since delivery. The 4% CHD-FA solution was a yellow/brown slightly viscous solution with a strong odour and a pH of 2.1 at 25° C.

1.2. Methods

1.2.1 Regulatory Issues

All animal experiments were performed under UK Home Office Licence 40/3101 Invasive Fungal Diseases (Licence Holder Dr Peter Warn) and with local ethical committee clearance. All experiments will be performed by technicians that have completed parts 1, 2 and 3 of the Home Office Personal License course and hold a current personal license. All experiments were performed in dedicated Biohazard 2 facilities within the Biological Services Unit of The University of Manchester (this site holds a Certificate of Designation).

1.2.2 Animal Model

Mice used in this study, male CD1 mice (an outbred strain that is very similar to Swiss mice) were supplied by Charles River (Margate UK) and were specific pathogen free (16-18 g at delivery). All mice weighed 20-22 g at the time of immunosuppression.

Mice were housed in individual ventilated cages (IVCs) that are supplied with HEPA filtered air. Sterile aspen chip bedding was supplied in pre-autoclaved boxes. Sterile water was provided ad libitum using disposable pouches. Standard mouse chow was provided ad libitum (food was moistened into mash if mice demonstrated signs of sepsis).

Mice experienced a 12 hour light dark cycle at 22±1° C., 55-60% relative humidity and background noise of <60 db.

Animals were treated using either 300 disposable ‘insulin’ Monojects (for iv or ip administrations) or reusable 19 G gavage needles.

All animals were immunosuppressed with a single dose of 200 mg/kg cyclophosphamide (Pharmacia) intraperitoneally (ip) 3 days before infection. This results in a profound state of neutropenia lasting for 3-4 days post infection.

1.2.3 Experiment Duration

The experiment was continued till 53 hours post infection.

1.2.4 Animal Group Size

For the combination study animals were treated in groups of 4 mice per treatment group.

1.2.5 Infection

Mice were infected with 0.2 ml of a suspension of FA7070 in PBS+0.05% tween 80 containing 1.5×10⁵ blastoconidia/ml i.e. 3.0×10⁴ yeasts per mouse. Following infection all mice were observed at least 4 times daily. Animals exceeding the severity band of the experiment were humanely euthanized. 1.2.6 Antifungal Treatment

Mice were treated 5 hours post infection with either:
a) 0.125 ml of 2% CHD-FA administered by gavage (assuming mice are 25 g at the time of treatment). CHD-FA was administered twice daily (total of 6 doses administered).
b) 0.125 ml of 0.5% CHD-FA administered by gavage (assuming mice are 25 g at the time of treatment). CHD-FA was administered twice daily (total of 6 doses administered).
c) 10 mg/kg fluconazole administered intravenously in 5% glucose (in 0.25 ml)
d) Combination therapy of 0.125 ml of 2% CHD-FA administered twice daily orally and 10 mg/kg fluconazole administered intravenously in 5% glucose.
e) Combination therapy of 0.125 ml of 0.5% CHD-FA administered twice daily orally and 10 mg/kg fluconazole administered intravenously in 5% glucose.
f) 0.5 mg/kg amphotericin B (diluted in 5% glucose) administered intraperitoneally.
g) Vehicle treated mice will be given 0.125 ml of 0.9% saline by gavage administered twice daily and 0.25 ml 5% glucose administered intravenously.

1.2.7 End of Animal Experiment

53 hours post infection all animals were euthanized using a schedule 1 procedure. All animals were weighed; kidneys were removed immediately and homogenized in ice cold sterile phosphate buffered saline. Kidney homogenates were quantitatively cultured onto Sabouraud Dextrose agar and incubated at 37° C. for up to 4 days and colonies counted.

1.2.8 Statistical Analysis

The data from the culture burdens was analyzed by the Kruskal-Wallis test using Stats Direct.

1.3 Results

1.3.1 Kidney Burdens

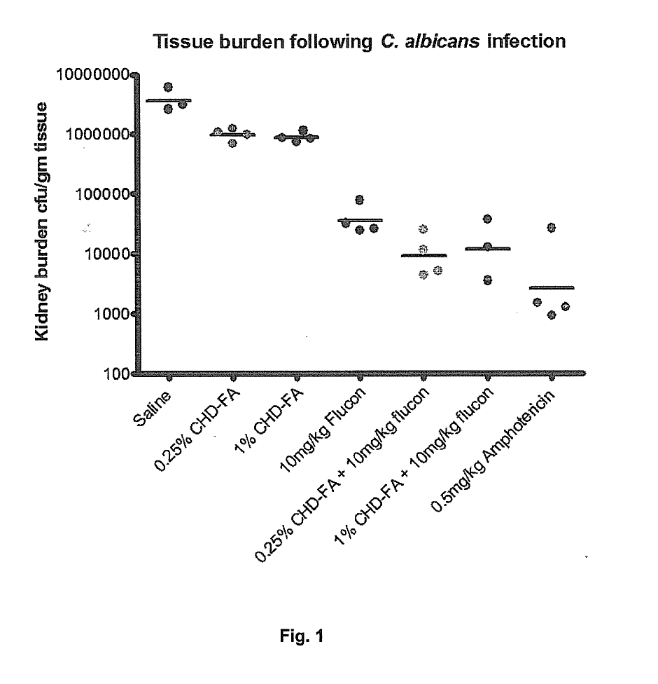

A summary of the kidney burdens is detailed in FIG. 1.

In this study there were no side effects noted after treatment with CHD-FA and study was ended at 53 hours post infection due to severe infection in the vehicle treated group.

1.3.2. Statistical Analysis

TABLE 1
Kruskal-Wallis: all pairwise comparisons (Conover-Inman)

| | | | | | 0.25% CHD- | | |
|---|---|---|---|---|---|---|---|
| | | | | | FA + | 1% CHD-FA + | |
| | | 0.25% | 1% CHD- | 10 mg/kg | 10 mg/kg | 10 mg/kg | 0.5 mg/kg |
| | Saline | CHD-FA | FA | Fluconazole | fluconazole | fluconazole | Amphotericin |
| Saline | | NS (0.06) | 0.028 | <0.0001 | <0.0001 | <0.0001 | <0.0001 |
| 0.25% CHD-FA | | | NS (0.67) | 0.0026 | <0.0001 | <0.0002 | <0.0001 |
| 1% CHD-FA | | | | 0.007 | <0.0001 | 0.006 | <0.0001 |
| 10 mg/kg | | | | | 0.044 | NS (0.19) | 0.004 |
| Fluconazole | | | | | | | |
| 0.25% CHD-FA + | | | | | | NS (0.51) | NS (0.29) |
| 10 mg/kg | | | | | | | |
| fluconazole | | | | | | | |
| 1% CHD-FA + | | | | | | | NS (0.11) |
| 10 mg/kg | | | | | | | |
| fluconazole | | | | | | | |
| 0.5 mg/kg | | | | | | | |
| Amphotericin | | | | | | | |

NS = not significant

1.4 Summary
Experiments were established using 0.125 ml gavages of 2% and 0.5% CHD-FA twice daily (equivalent to 1% and 0.25% CHD-FA administered at 10 ml/kg).
5 ml/kg of 2% or 0.5% CHD-FA (equivalent to 10 ml/kg at 1% and 0.25% CHD-FA) was well tolerated in mice.
5 ml/kg of 2% or 0.5% CHD-FA (equivalent to 10 ml/kg at 1% and 0.25% CHD-FA) was effective at reducing the kidney burden on mice infected with Candida albicans. The burden following treatment was significantly lower than vehicle treated mice (˜0.6 log₁₀ cfu/gram reduction)
5 ml/kg of 2% or 0.5% CHD-FA (equivalent to 10 ml/kg at 1% and 0.25% CHD-FA) was additive when used in combination with fluconazole. The combined reduction in tissue burden was significantly superior to either treatment used as monotherapy.

### EXAMPLE 2

### In Vitro Efficacy of CHD-FA Against Aspergillus and Zygomycetes

2.1 Physical Properties of CHD-FA Also Known as Fulvic Acid

CHD-FA was reconstituted as a 4% solution. The solutions were stored at room temperature in the dark since delivery. The 4% CHD-FA solution was a yellow/brown slightly viscous solution with a strong odour and a pH of 2.1 at 25° C.

2.2 Methods—Preliminary Experiment

2.2.1 Fungal Isolates

Susceptibility tests were performed on the following isolates which are all strains isolated from cases of human clinical disease.
(i) 2×Aspergillus fumigatus.
(ii) 2×Aspergillus terreus—these strains are moderately resistant in vitro and resistant in vivo to amphotericin B which is typical of A. terreus strains.
(iii) 2×Aspergillus flavus—these strains are of intermediate susceptibility in vitro and respond poorly in vivo to amphotericin B (1 strain).
(iv) 2×Aspergillus flavus—these strains are of intermediate susceptibility in vitro and respond poorly in vivo to amphotericin B (1 strain).
(v) 2×Absidia corymbifera—these strains are susceptible in vitro but respond very poorly in vivo to amphotericin B.
(vi) 2×Fusarium solani—these strains are highly resistant in vitro and do not respond in vivo to amphotericin B.

Each culture was grown on Sabouraud agar at 37° C. for 10 days to ensure purity and to allow spores to mature

2.2.2 Media

RPMI-1640 (Sigma, Dorset, UK) supplemented to 2% glucose (Sigma), buffered with morpholinopropanesulfonic acid (MOPS), (Sigma) and adjusted to pH 7.0 was used as recommended in the Clinical Laboratory Standards M38A document (Reference method for broth dilution antifungal susceptibility testing of conidium-forming filamentous fungi. Approved standard. Document M38-A 2002a. NCCLS, Wayne, Pa. 2002. NCCLS, Wayne, Pa., USA).

2.2.3 Preparation of the Inoculum

a) All fungi were cultured in ambient air at 35-37° C. on recovery medium (Sabouraud dextrose agar) for 8-10 days before testing.

b) Inoculum suspensions were prepared from day 8 to 10 day cultures grown on Sabouraud dextrose agar at 37° C. in vented tissue culture flasks to avoid crosscontamination. Spores were harvested by flooding the surface of the growth with 25 ml of sterile phosphate buffered saline plus 0.05% Tween 80. The spore count was adjusted using a counting chamber.

c) The inoculum was completely suspended by vigorous shaking on a vortex mixer for 15 s. The final spore density in the MIC tests was between 0.5×104 and 5×104 CFU/mL as demonstrated by quantitative colony counts. Drug-free and cell-free controls were included. (The RPMI medium used in the plates was prepared at either 2× or 4×the final strength to allow for dilution once the inoculum and diluted CHD-FA was added).

2.2.4 Assay Conditions

Sterile plastic, disposable, microtitration plates with 96 flat-bottom wells were used.

Step 1 Addition of Amphotericin B (Stock Solution Prepared in 100% DMSO)

a) Column 1 of the microtitration tray was filled with 100 μL of sterile water containing four times the final drug concentration (16 mg/L amphotericin B).

b) Columns 2-12 were filled with 50 μL of distilled water

c) 50 μL amounts were taken from wells in column 1 and diluted two-fold by transferring them to column 2 with a multichannel pipette (±2% coefficient of variation). 50 μL samples were then removed from column 2 and transferred to column 3, and so on through to column 10. The last 50 μL of diluted drug is then discarded. Thus, each well in columns 1-10 will contain 50 μL of water containing four times the final antifungal drug concentrations.

Step 2 Addition of CHD-FA

Stock solutions of CHD-FA were produced containing 4%, 2%, 1%, 0.5% and 0.25% of the native compound.

100 μL of the diluted CHD-FA was added to microtitration trays in so that row A contains a final dilution of 2% row B, 1%, row C 0.5%, row D 0.25% row E 0.125% and row F diluent only.

Step 3 Addition of Aspergillus or Zygomycetes Strains

50 μL of the diluted spore suspension in 4×RPMI was added to all wells. This produces a well containing 200 μL final volume (made up of 50 μL diluted antifungal, 100 μL diluted CHD-FA or diluent, 50 μL of 4×RPMI containing fungal spores)

Step 4 Incubation of Plates

All plates were incubated at 37° C. in air in a darkened incubator.

Step 5 Reading of Plates

Plates were read visually with the endpoint taken as the lowest concentration of drug that inhibited growth by 50% of that of the drug free control.

2.3. Results—Preliminary Experiment

2.3.1 MICs Against CHD-FA and Amphotericin B

MICs against the single agents demonstrated that CHD-FA at 4%, 2% and 1% inhibited the growth of Aspergillus and Zygomycetes for at least 24 hours. The MIC values for CHD-FA and Amphotericin B are detailed in Table 2.

TABLE 2
Efficacy of CHD-FA against Aspergillus and zygomycetes

| Isolate | Isolate | MIC | Amphotericin |
|---|---|---|---|
| Species | Number | (%) CHD-FA | MIC (mg/L) |
| A. fumigatus | 1 | 0.5 | 0.5 |
| A. fumigatus | 2 | 0.5 | 0.25 |
| A. terreus | 1 | 0.5 | 0.5 |
| A. terreus | 2 | 0.5 | 1.0 |
| A. flavus | 1 | 0.5 | 4.0 |
| A. flavus | 2 | 0.25 | 0.5 |
| Absidia | 1 | 0.25 | 0.25 |
| Absidia | 2 | 0.25 | 0.06 |
| Fusarium | 1 | 0.25 | 2.0 |
| Fusarium | 2 | 0.25 | 2.0 |

3.3.2 MICs Against the Combination of CHD-FA and Amphotericin B

MICs against the combination of CHD-FA and amphotericin B are shown in table 3. It is of note than none of the combinations were antagonistic but the combination was highly effective against a strain of Aspergillus fumigatus that is resistant in vitro and in vivo to amphotericin B.

TABLE 3
Efficacy of the combination of CHD-FA and amphotericin B against Aspergillus

| Isolate | CHD-FA | MIC | Amphotericin | Combination | |
|---|---|---|---|---|---|
| Species | Dilution | CHD-FA | MIC | MIC | Effect |
| A. fumigates | 0.5 | No Growth | No Growth | No Growth | possible |
| strain 1 | 0.25 | No Growth | No Growth | No Growth | synergy |
| | 0.125 | Growth | 0.06 | <0.06 | |
| A. fumigates | 0.5 | No Growth | No Growth | No Growth | neither |
| strain 2 | 0.25 | No Growth | No Growth | No Growth | synergy nor |
| | 0.125 | Growth | 0.125 | 0.125 | antagonism |
| A. terreus | 0.5 | No Growth | No Growth | No Growth | neither |
| strain 1 | 0.25 | No Growth | No Growth | No Growth | synergy nor |
| | 0.125 | Growth | 0.25 | 0.25 | antagonism |
| A. terreus | 0.5 | No Growth | No Growth | No Growth | neither |
| strain 2 | 0.25 | No Growth | No Growth | No Growth | synergy nor |
| | 0.125 | Growth | 0.125 | 0.125 | antagonism |
| A. flavus | 0.5 | No Growth | No Growth | No Growth | combination |
| strain 1 | 0.25 | No Growth | No Growth | No Growth | reduced MIC |
| | 0.125 | Growth | >4.0 | <0.06 | from resistant |
| | | | | | to highly |
| | | | | | susceptible |
| A. flavus | 0.5 | No Growth | No Growth | No Growth | neither |
| strain 2 | 0.25 | No Growth | No Growth | No Growth | synergy nor |
| | 0.125 | Growth | 0.25 | 0.5 | antagonism |

2.4. Summary
CHD-FA does not demonstrate antagonistic activity when used in combination with amphotericin B Aspergillus spp.
The combination of 0.25% CHD-FA with amphotericin B (0.06-0.5 mg/l) inhibits the growth of all Aspergillus isolates tested regardless of the amphotericin B MIC of the isolates.

### EXAMPLE 3

### In Vitro Efficacy of a Combination of Fluconazole and CHD-FA Against Candida Spp.

3.1 Physical Properties of CHD-FA Also Known as Fulvic Acid

CHD-FA was reconstituted as a 4% solution. The solutions were stored at room temperature in the dark since delivery. The 4% CHD-FA solution was a yellow/brown slightly viscous solution with a strong odour and a pH of 2.1 at 25° C.

3.2 Methods—Preliminary Experiment

3.2.1 Fungal Isolates

Susceptibility tests were performed on 5 isolates of Candida albicans all clinical isolates (all with reduced susceptibility to fluconazole). Each culture was grown on Sabouraud agar at 37° C. for 48 h to ensure purity.

3.2.2 Media

RPMI-1640 (Sigma, Dorset, UK) supplemented to 2% glucose (Sigma), buffered with morpholinopropanesulfonic acid (MOPS), (Sigma) and adjusted to pH 7.0 was used as recommended in the European Committee on Antimicrobial Susceptibility Testing (E.Dis. 7.1) (Rodriguez-Tudela, J. L., F. Barchiesi, J. Bille, E. Chryssanthou, M. Cuenca-Estrella, D. Denning, J. P. Donnelly, B. Dupont, W. Fegeler, C. Moore, M. Richardson, P. E. Verweij, and the Subcommittee on Antifungal Susceptibility Testing (AFST) of the ESCMID European Committee for Antimicrobial Susceptibility Testing (EUCAST) 2003. Method for the determination of minimum inhibitory concentration (MIC) by broth dilution of fermentative yeasts. Clinical Microbiology and Infection 9:1-VIII.)

3.2.3 Preparation of the Inoculum

a) All yeasts were cultured in ambient air at 35-37° C. on recovery medium (Sabouraud dextrose agar) for 18-24 h before testing.

b) The inoculum was prepared by picking five distinct colonies from 18 to 24 h cultures and suspending them in 5 mL of sterile distilled water.

c) The inoculum was completely suspended by vigorous shaking on a vortex mixer for 15 s. The cell density was adjusted to the density of a 0.5 McFarland standard by adding sterile distilled water and measuring absorbance in a spectrophotometer at a wavelength of 530 nm. This gave a yeast suspension of 1-5×10⁶ cfu/mL. A working suspension was prepared by a further dilution of 1 in 10 further in 4×RPMI to give 1-5×10⁶ cfu/mL (The RPMI medium used in the plates was prepared at 4×the final strength to allow for a 75% dilution once the inoculum and diluted CHD-FA was added).

3.2.4 Assay Conditions

Sterile plastic, disposable, microtitration plates with 96 flat-bottom wells were used.

Step 1 Addition of Fluconazole

a) Column 1 of the microtitration tray was filled with 100 μL of sterile water containing four times the final drug concentration (512 mg/L fluconazole).

b) Columns 2-12 were filled with 50 μL of distilled water

c) 50 μL amounts were taken from wells in column 1 and diluted two-fold by transferring them to column 2 with a multichannel pipette (±2% coefficient of variation). 50 μL samples were then removed from column 2 and transferred to column 3, and so on through to column 10. The last 50 μL of diluted drug is then discarded. Thus, each well in columns 1-10 will contain 50 μL of water containing four times the final antifungal drug concentrations.

Step 2 Addition of CHD-FA

Stock solutions of CHD-FA were produced containing 4%, 2%, 1%, 0.5% and 0.25% of the native compound. 100 μL of the diluted CHD-FA was added to microtitration trays in so that row A contains a final dilution of 2% row B, 1%, row C 0.5%, row D 0.25% row E 0.125% and row F diluent only.

Step 3 Addition of Candida Albicans

50 μL of the diluted Candida albicans suspension in 4×RPMI was added to all cells. This produces a well containing 200 μL final volume (made up of 50 μL diluted fluconazole, 100 μL diluted CHD-FA or diluent, 50 μL of 4×RPMI containing Candida albicans)

Step 4 Incubation of Plates

All plates were incubated at 37° C. in air in a darkened incubator.

Step 5 Reading of Plates

Plates were read visually with the endpoint taken as the lowest concentration of drug that inhibited growth by 50% of that of the drug free control.

3.3.1 Results—Preliminary Experiment

Initial tests demonstrated that CHD-FA at 4%, 2% and 1% inhibited the growth of Candida albicans for at least 24 hours when combined with 1×RPMI 1640 culture medium, growth in 0.5% CHD-FA occurs at similar levels to solvent controls. The inhibition of growth (4%, 2% and 1%) was possibly due to the strongly acidic pH. At the native pH of CHD-FA (pH 2.1) no synergy or antagonism with fluconazole could be detected.

The pH of CHD-FA was adjusted to pH7.0 using 10M NaOH solution (the ideal pH for fluconazole activity). This resulted in a brown slightly viscous solution with a strong characteristic odour. Susceptibility assays were repeated as above.

As previously noted with native CHD-FA, 4%, 2% and 1% solutions inhibited the growth of Candida albicans for at least 24 hours when combined with 1×RPMI 1640 culture medium, growth in 0.5% CHD-FA occurs at similar levels to solvent controls. No synergy or antagonism with fluconazole could be detected.

The MICs of the Candida albicans strains with and without CHD-FA are listed in Table 4 (the pH of CHD-FA was adjusted to 7.0).

TABLE 4
Minimum Inhibitory Concentrations of Fluconazole (mg/L) combined with
CHD-FA

| | 2% | 1% | | | 0.125% | |
|---|---|---|---|---|---|---|
| Candida | CHD- | CHD- | 0.5% | 0.25% | CHD- | |
| Strain | FA | FA | CHD-FA | CHD-FA | FA | Solvent |
| 1 | No | No | 16 | 16 | 16 | 128 |
| | growth | growth | | | | |
| 2 | No | No | 32 | 16 | 16 | 16 |
| | growth | growth | | | | |
| 3 | No | No | 2 | 16 | 16 | 16 |
| | growth | growth | | | | |
| 4 | No | No | 32 | 32 | 32 | 32 |
| | growth | growth | | | | |
| 5 | No | No | 32 | 32 | 16 | 16 |
| | growth | growth | | | | |

3.4 Methods—Main Experiment

3.4.1 Fungal Isolates

Susceptibility tests were performed on 40 clinical isolates of Candida of which 38 had reduced susceptibility to fluconazole. The group comprised 25 C. albicans, 11 C. glabrata (all with reduced susceptibility to fluconazole), and 4 C. tropicalis (all with reduced susceptibility to fluconazole). Each culture was grown on Sabouraud agar at 37° C. for 48 h to ensure purity.

3.4.2 Media

As indicated in 3.2.2.

3.4.3 Preparation of the Inoculum

As indicated in 3.2.3.

3.4.4 Assay Conditions

Sterile plastic, disposable, microtitration plates with 96 flat-bottom wells were used.

Step 1 Addition of Fluconazole

a) Column 1 of the microtitration tray was filled with 100 μL of sterile water containing four times the final drug concentration (512 mg/L fluconazole).

b) Columns 2-12 were filled with 50 μL of distilled water

c) 50 μL amounts were taken from wells in column 1 and diluted two-fold by transferring them to column 2 with a multichannel pipette (±2% coefficient of variation). 50 μL samples were then removed from column 2 and transferred to column 3, and so on through to column 10. The last 50 μL of diluted drug is then discarded. Thus, each well in columns 1-10 will contain 50 μL of water containing four times the final antifungal drug concentrations.

Step 2 Addition of CHD-FA

Stock solutions of CHD-FA were produced containing 4% and 2% (also 1% and 0.5% for Candida parapsilosis and Candida krusei). 100 μL of the diluted CHD-FA was added to microtitration trays in so that rows contain final dilutions of:
For Candida albicans and tropicalis: 1st row of pair 0.5% CHD-FA, 2nd row of pair solvent only
For Candida glabrata: 1st row of triplet 1% CHD-FA, 2nd row of triplet 0.5% CHD-FA, 3rd row of triplet solvent only
For Candida parapsilosis and Candida krusei rows included 2%, 1%, 0.5%, 0.25 and 0.125% CHD-FA and a row of solvent only.

All the above concentrations were mixed with fixed concentrations of fluconazole at 128, 32, 8, 2, 0.5 mg/L and solvent for synergy/antagonism assessment.

Step 3 Addition of Candida Spp

50 μL of the diluted Candida spp suspension in 4×RPMI was added to all wells. This produces a well containing 200 μL final volume (made up of 50 μL diluted fluconazole, 100 μL diluted CHD-FA or diluent, 50 μL of 4×RPMI containing Candida)

Step 4 Incubation of Plates

All plates were incubated at 37° C. in air in a darkened incubator.

Step 5 Reading of Plates

Plates were read visually with the endpoint taken as the lowest concentration of drug that inhibited growth by 50% of that of the drug free control.

3.5. Results—Main Experiment

Results are summarized in Tables 5 to 10.

No regrowth of Candida occurred on prolonged incubation (96 hours) at 37° C. or room temperature.

TABLE 5
Efficacy of CHD-FA (0.5%) in combination with
fluconazole against Candida albicans

| Isolate | Isolate | Fluconazole MIC | Fluconazole MIC |
|---|---|---|---|
| Species | Number | (mg/L) alone | with 0.5% CHD-FA |
| C. albicans | 1 | 128 | No Growth |
| C. albicans | 2 | 8 | No Growth |
| C. albicans | 3 | 16 | No Growth |
| C. albicans | 4 | 4 | No Growth |
| C. albicans | 5 | 128 | No Growth |
| C. albicans | 6 | >128 | No Growth |
| C. albicans | 7 | 128 | No Growth |
| C. albicans | 8 | 128 | No Growth |
| C. albicans | 9 | 64 | No Growth |
| C. albicans | 10 | 128 | No Growth |
| C. albicans | 11 | 0.5 | No Growth |
| C. albicans | 12 | 64 | No Growth |
| C. albicans | 13 | 0.5 | No Growth |
| C. albicans | 14 | 32 | No Growth |
| C. albicans | 15 | 2 | No Growth |
| C. albicans | 16 | 64 | No Growth |
| C. albicans | 17 | >128 | No Growth |
| C. albicans | 18 | >128 | No Growth |
| C. albicans | 19 | 0.5 | No Growth |
| C. albicans | 20 | >128 | No Growth |
| C. albicans | 21 | 4 | No Growth |
| C. albicans | 22 | 2 | No Growth |
| C. albicans | 23 | 1 | No Growth |
| C. albicans | 24 | 1 | No Growth |
| C. albicans | 25 | >128 | No Growth |

TABLE 6
Efficacy of CHD-FA (0.5%) in combination with
fluconazole against Candida glabrata

| Isolate | Isolate | Fluconazole MIC | Fluconazole MIC |
|---|---|---|---|
| Species | Number | (mg/L) alone | with 0.5% CHD-FA |
| C. glabrata | 1 | >128 | >128 |
| C. glabrata | 2 | >128 | >128 |
| C. glabrata | 3 | >128 | >128 |
| C. glabrata | 4 | >128 | >128 |
| C. glabrata | 5 | >128 | Trace Growth |
| C. glabrata | 6 | >128 | >128 |
| C. glabrata | 7 | >128 | >128 |
| C. glabrata | 8 | >128 | >128 |
| C. glabrata | 9 | >128 | >128 |
| C. glabrata | 10 | >128 | >128 |
| C. glabrata | 11 | >128 | >128 |

TABLE 7
Efficacy of CHD-FA (1%) in combination with
fluconazole against Candida glabrata

| Isolate | Isolate | Fluconazole MIC | Fluconazole MIC |
|---|---|---|---|
| Species | Number | (mg/L) alone | with 0.5% CHD-FA |
| C. glabrata | 1 | >128 | No Growth |
| C. glabrata | 2 | >128 | No Growth |
| C. glabrata | 3 | >128 | No Growth |
| C. glabrata | 4 | >128 | No Growth |
| C. glabrata | 5 | >128 | No Growth |
| C. glabrata | 6 | >128 | No Growth |
| C. glabrata | 7 | >128 | No Growth |
| C. glabrata | 8 | >128 | No Growth |
| C. glabrata | 9 | >128 | No Growth |
| C. glabrata | 10 | >128 | No Growth |
| C. glabrata | 11 | >128 | No Growth |

TABLE 8
Efficacy of CHD-FA (0.5%) in combination with
fluconazole against Candida tropicalis

| Isolate | Isolate | Fluconazole MIC | Fluconazole MIC |
|---|---|---|---|
| Species | Number | (mg/L) alone | with 0.5% CHD-FA |
| C. tropicalis | 1 | 16 | No Growth |
| C. tropicalis | 2 | >64 | No Growth |
| C. tropicalis | 3 | 16 | No Growth |
| C. tropicalis | 4 | 16 | No Growth |

TABLE 9
Efficacy of CHD-FA (0.5%) in combination with
fluconazole against Candida parapsilosis

| | | Fluconazole | Fluconazole | CHD-FA (%) |
|---|---|---|---|---|
| Isolate | Isolate | MIC (mg/L) | MIC with 0.5% | without |
| Species | Number | alone | CHD-FA | fluconazole |
| C. parapsilosis | 1 | 8 | No Growth | 0.25 |
| C. parapsilosis | 2 | 2 | No Growth | 0.5 |
| C. parapsilosis | 3 | 8 | No Growth | 0.5 |
| C. parapsilosis | 4 | 8 | No Growth | 0.5 |
| C. parapsilosis | 5 | 8 | No Growth | 0.5 |

TABLE 10
Efficacy of CHD-FA (0.5%) in combination with
fluconazole against Candida krusei

| | | Fluconazole | Fluconazole | CHD-FA (%) |
|---|---|---|---|---|
| Isolate | Isolate | MIC (mg/L) | MIC with 0.5% | without |
| Species | Number | alone | CHD-FA | fluconazole |
| C. krusei | 1 | 32 | No Growth | 0.25 |
| C. krusei | 2 | 32 | No Growth | 0.5 |
| C. krusei | 3 | 32 | No Growth | 0.25 |
| C. krusei | 4 | 32 | No Growth | 0.5 |
| C. krusei | 5 | 32 | No Growth | 0.5 |

3.6 Summary
CHD-FA is equally effective as an antifungal agent in vitro whether examined at it's native pH of 2.1 or buffered to pH7.0.
CHD-FA inhibits the growth of Candida albicans, Candida tropicalis, Candida parapsilosis and Candida krusei when used as a 0.5% in vitro.
CHD-FA inhibits the growth of Candida glabrata when used as a 1% solution.
The combination of 1% CHD-FA with fluconazole (0.25-128 mg/l) inhibits the growth of all Candida isolates tested.
CHD-FA is highly effective against strains of C. krusei that are intrinsically resistant to fluconazole.

1-26. (canceled)

27. A composition comprising a combination of:
a) fulvic acid, or a salt, ester, or derivative thereof, and
b) an antifungal compound selected from the group consisting of fluconazole and amphotericin B.

28. The composition according claim 27, wherein the fulvic acid is carbohydrate-derived fulvic acid (CHD-FA).

29. The composition according to claim 27, wherein the salt comprises a sodium or potassium salt.

30. The composition according to claim 27, comprising about 10 ml/kg of a solution of about 0.25% to about 1% (w/v) of fulvic acid, or a salt, ester, or derivative thereof, and about 10 mg/kg of fluconazole.

31. The composition according to claim 27, comprising a solution of about 0.25% (w/v) fulvic acid, or a salt, ester, or derivative thereof, and from about 0.06 mg/l to about 0.5 mg/l of amphotericin B.

32. A pharmaceutical composition comprising the combination according to claim 27 as active ingredients.

33. The pharmaceutical composition according to claim 32, in the form of a liquid, tablet, or capsule.

34. The pharmaceutical composition according to claim 32, wherein the composition is in a form suitable for oral administration.

35. The pharmaceutical composition according to claim 32, wherein the composition is in a form suitable for topical administration.

36. The pharmaceutical composition according to claim 35, in the form of a cream, ointment, or liquid.

37. A method of treatment or prophylaxis of a disease or condition of the human or non-human animal body, comprising administering to a subject in need thereof, a therapeutically effective amount of the composition according to claim 27.

38. The method according to claim 37, wherein the disease or condition is caused by a drug-resistant fungus.

39. The method according to claim 37, wherein the subject in need thereof is immunosuppressed or immunocompromised.

40. The method according to claim 37, wherein the administration is oral, topical, intravenous or intraperitoneal.
